SEQUENCE LISTING (15 sequences)

SEQ ID NO: 1 - DNA, 4177 bp, Gallus gallus
  CDS (1409)..(2878)
  modified_base (1119): a, t, c or g
cgacagacttgaggggttctctgccaactgatctctcaccgcaatgggtagacggatctctacgtggagactgatctctcaccacgacacgagcttcctgccttccgatcctcctctacggaccgtttgctgacggacttccctgggcctgctacctgagacctgctgcttcctccctgacctgcatcctctcgctgccccagaccggcctcgctgctcctgcccttcggcctcggaccgtcggaacatcgtgcaacgggactgctgccggatcctggtggtgactatccccgctttacgcaattcttgcctctttctatcttttctatcgctcgccttcccttccccatcaccccaatccttaatagcgtccgtcctcccctttccccatctcccttattaacatttgtaataaactggtcggaccaacatttgaaccgctgtttcttaatctcacgccgggcatacatatttcaaagaacctcttctccctcctataaattggagcgagacattttttatggcgtagtcggcaggatacccgccgtgagagtgttgtccttccagataatagtctgaaactttctgcgtgtacctcctggagttgcaagaagcgatacttcttgataacttagacgtgagcacctctccaggaagatcgcttcatactctgaaactttactatttatgtgtgtacctctcgaggatgtatgaattttgtctaattgtatttatttaatacgtgtgtgcctcctcgggaagacctctctgcattttgtgaacccctctctacgtgtgcgcctcttggggaagcaagatacacgttttttgacttaaaaaacttgtgtgcctcccaagaagttttctcactttgctgaaaattgtttatgtatgcacctctcgaggacgtatgaatcttgtctaattgcatttaatacgtgtgtgcctcctcgggaagacctctctgcattttgtgacttaaggatcttgcaacttaagtgtgaaatttgaacctctttcgtgcgtgcctcttggggaagtgaggaagtgatacacgttttttgatttaaaaaacgtgtgcgcttctccaagaagtttattcactttgttaatcctagnaaagtgttgttttagcttaaaattaactgtgggttttgaaaccgaagtgtgccttgctttggtgtggtgtttgcagttttttgtgtggcttcgcagggaagttaggagcgattttaagttggtttagtctctttgcccttgtgctttcctcaacaaagggaggcgcaatcggaacatttacatttctttagttgtggtgtgcctccgtgggagaggcgataaggagttatttgtacttttgaataggagtacctcctctctcagtgtatatctttctgtgtatttgggaatgagcaacagtatggccagtatgaaaagtgaagatgtattatttgatcttttagaaaagcatggtgctcggccttctgtatcaggggtggattgggcacgacagaactggtataatttgcaaagtgtttcagaccgtattcgtgttttacaaaatgaggctcgtactcgggccggaaaagggaaatcttttatttgtgcagtactcggtgctgctttaaaagcagctgtggagttccgagaggaaaagaactctacggaaacccagagtattcaagcattacaggaatcggttaaagtgacgcaagaattggtaaaatctctgcaaagccaaataaggagtcttgaggatcaattagaaagagaaaaacacaattcggttctgttgcaaacagcttttaaggagctgataacgtgtaaggacaccggtgacactgttatccacagtgcacctcaagaaaaagtttatcctcaagggaaattacaagaggtgaaggaaaggctagataaattagaggcctctccagcccacattcgtcctttgataaaaactgaatatactttcgataacagtgagaatctagatcctcaaatgaatgttaaggaaattcccttttcggccactgaactggccaaactgaaaaaggatttcagtcgctccccaaaggagtctgaaacagagtacgtctggagagttagtctcactggcggagaccagatcctactaacagagaaagaagctgaaggttactggggaccaggagtatttttaaccactggcaataatcgtgctccctggtccttaacacagagggctgcctattgggcagggggtctcaaccctttagaaaggggggaccctcttgctattactggaactatcgaccagttagtggagaatgttcagaaagctgcttgtctccaaatgatgtatgatagaaagttgcagccacataatgaatcacccatgatgttacctgttaatccggagagactgacacctctaatcaggggacttcctgaatcgttaaaacctataggtatacaactccaaggaaagatacaagccatgtctcagggagagagaacctgggcagcgttggagggatctgtagcccctaaccaccagtcaggacccaaagtgtggacttggggagaggttgcccaagaattaattaactatggaagaaaatatgggccggtggtttctacctgcagtaaatttgagccaagaggagtaaggcttgcagtagccagccttgcctccaggcctcctagcccaagacttattggaaccaaaaaggtttcatccccagtaaaaacggggacacgatgcattgatcataaacgcaatggactttggacnctgggctggacaaagggtattccacgagatttgatgaatggattacccacagtcagattagagaaattagttaactgctggccagaacaaaagctcaaggggagctgatgccttcgcccccccctcccaggtgagcgggaggtgggtgggggggtgaagggtggatgtttattaggaagctcacgactaaaggaaacaatctgttaattgtttatttattattagtggttattgtcaaatgtacggttgtctcttttctctcttctattcattatgtaatattcatgttaccactcctgaagaatcacggggtggtgtctatggcaagttgcattgtgtactgttgcaactcttatgtttgtatgattccatgttttatacaagatgttgtatcccctatttactttgtaaccaaacctgaaaaatgtttgtaatgattgtatgaaacatttgattccacaacccctccctcctttacccttgtgcttgctatcttctctcaccaccatggatgcccagtgtccaatttttaagcaacctttgagtcacggggtggtgtaagagactattcttttatatcattgactcaaagtttgctgaggaacaagtccaggcaagtcctgggcaaaggcagagaaatcttttgtcttgaggacactgatggacaggtcctggctaaggattgtgaaatcctttaaggagcacagatggacaaggccaggggcatcgagagagagataagctgccgctaatggccgggaaacggtctttttgtgtggacttatctcaaggaaaatggccatctcaggaggtatgcacaggactcttgctcaagcccccaggaatgtcacgtaggcagcagaaaatggaggataaaagaggtccaataaccacaacggtggaagctgatccttcaccacaaccacggcaacgggagangcttatctctcaccacgacagacttgaggggttctctgccaactgatctctcaccgcaatgggtagacggatctctacgtggagactgatctctcaccacgacacgagcttcctgccttccgatcctcctctacggaccgtttgctgatggacttccctgggcctgctacctgagacctgctgcttcctccctgacctgcatcctctcgctgccccagaccggcctcgctgctcctgcccttccgtcggaacatcgtgcaacgggactgctgccggatcctggtggtgactatccccgcttaacgcaattctngcctctttctatcttttntatcgctcgccttcccttccccatcaccccaatccttaatagcgtccgtcctcccctttccccatctcccttattaacatttgtaataaactggtcggaccaacaaaaaaaaaaaaaaaaaaaaaaaaaaaaaa

SEQ ID NO: 2 - protein, 490 aa, Gallus gallus
MSNSMASMKSEDVLFDLLEKHGARPSVSGVDWARQNWYNLQSVSDRIRVLQNEARTRAGKGKSFICAVLGAALKAAVEFREEKNSTETQSIQALQESVKVTQELVKSLQSQIRSLEDQLEREKHNSVLLQTAFKELITCKDTGDTVIHSAPQEKVYPQGKLQEVKERLDKLEASPAHIRPLIKTEYTFDNSENLDPQMNVKEIPFSATELAKLKKDFSRSPKESETEYVWRVSLTGGDQILLTEKEAEGYWGPGVFLTTGNNRAPWSLTQRAAYWAGGLNPLERGDPLAITGTIDQLVENVQKAACLQMMYDRKLQPHNESPMMLPVNPERLTPLIRGLPESLKPIGIQLQGKIQAMSQGERTWAALEGSVAPNHQSGPKVWTWGEVAQELINYGRKYGPVVSTCSKFEPRGVRLAVASLASRPPSPRLIGTKKVSSPVKTGTRCIDHKRNGLWTLGWTKGIPRDLMNGLPTVRLEKLVNCWPEQKLKGS

SEQ ID NO: 3 - DNA, 22 bp, Artificial Sequence
  Description of Artificial Sequence Primer
tggagtgacggcagttatctgg

SEQ ID NO: 4 - DNA, 22 bp, Artificial Sequence
  Description of Artificial Sequence Primer
ggcttcatccaccacatacagg

SEQ ID NO: 5 - DNA, 25 bp, Artificial Sequence
  Description of Artificial Sequence Primer
ccgtgcatctgccagtttgagggga

SEQ ID NO: 6 - DNA, 44 bp, Artificial Sequence
  Description of Artificial Sequence Primer adaptor
ctaatacgactcactatagggctcgagcggccgcccgggcaggt

SEQ ID NO: 7 - DNA, 27 bp, Artificial Sequence
  Description of Artificial Sequence Primer
ccatcctaatacgactcactatagggc

SEQ ID NO: 8 - DNA, 20 bp, Artificial Sequence
  Description of Artificial Sequence Primer
gggatccgccatgtcacaga

SEQ ID NO: 9 - DNA, 44 bp, Artificial Sequence
  Description of Artificial Sequence Primer
actatcgattctggaaccttcagaggtttttttttttttttttt

SEQ ID NO: 10 - DNA, 21 bp, Artificial Sequence
  Description of Artificial Sequence Primer
gtcgtgcaacgggactgcctg

SEQ ID NO: 11 - DNA, 25 bp, Artificial Sequence
  Description of Artificial Sequence Primer
ctatcgattctggaaccttcagagg

SEQ ID NO: 12 - DNA, 171 bp, Gallus gallus
tccttctctacggaccgtttgctgacggacttccctgggcctgctacctgagacctgctgcttcctccctgacctgcaccctctcgttgcccaagaccggcctcactgctcctgcccttcggcctcggaccatcggaacgtcgtgcaacgggactgctgctgaatcctggt

SEQ ID NO: 13 - DNA, 18 bp, Artificial Sequence
  Description of Artificial Sequence Primer
agaccggcctcactgctc

SEQ ID NO: 14 - DNA, 21 bp, Artificial Sequence
  Description of Artificial Sequence Primer
ggatctagatcctcaaatgaa

SEQ ID NO: 15 - DNA, 19 bp, Artificial Sequence
  Description of Artificial Sequence Primer
aattcttgggcaacctctc

(19) United States
(12) Patent Application Publication
(10) Pub. No.: US 2004/0072169 A1
(43) Pub. Date: Apr. 15, 2004

(54) Modified es cells and es cells-specific gene

(76) Inventors: Herve Acloque, Lyon (FR); Anne-Marie Birot, Sainte-Foy-les-Lyon (FR); Valerie Risson, Villeurbanne (FR); Bertrand Pain, Lyon (FR); Jacques Samarut, Villeurbanne (FR)

Correspondence Address: HELLER EHRMAN WHITE & MCAULIFFE LLP, 1666 K STREET,NW, SUITE 300, WASHINGTON, DC, 20006, US

(21) Appl. No.: 10/275,906

(22) PCT Filed: Apr. 19, 2001
(86) PCT No.: PCT/FR01/01207

(30) Foreign Application Priority Data
May 11, 2000 (FR) 00 06029

(51) Int. Cl. C12Q 1/68; A01K 67/027; C07H 21/04; C07K 14/465; C07K 16/18

(52) U.S. Cl. 435/6; 435/69.1; 435/320.1; 435/325; 435/366; 530/350; 530/388.1; 800/18; 536/23.5

(57) ABSTRACT

The invention concerns modified avian ES cells, specifically expressing an exogenous gene when they have a pluripotent character. The invention also concerns a nucleic acid and a polypeptide specifically expressed in pluripotent avian cells, and methods for detecting the pluripotent character of cells using said nucleic acid and polypeptide.

The present invention relates to modified avian ES cells specifically expressing an exogenous gene when they are pluripotent in nature. The invention also relates to a nucleic acid, and a polypeptide, expressed specifically in pluripotent avian cells, and to methods for detecting the pluripotent nature of cells using this nucleic acid and this polypeptide.

ES cells are pluripotent cells isolated from a very early embryo, which are capable of participating in the morphogenesis of all tissues, including germinal tissue, after they have been transplanted into host embryos. These cells were first of all isolated in mice, where they are very widely used to create mutant animals carrying highly targeted modifications of their genome. ES cells have been isolated and characterized in birds (Pain et al., 1996). These cells can be used to modify the genetic inheritance of the chicken (Etches et al., 1996, Pain et al., 1999). A culture medium which makes it possible to maintain the pluripotent nature of these avian cells was the subject of patent application Wo 96/12793.

The difficulty encountered by all those who wish to isolate ES cells in culture concerns the rapid identification of these cells and of their pluripotent nature. Several cellular markers have been used, such as the expression of alkaline phosphatase activity (Strickland et al., 1980), the expression of antigenic epitopes (Kemler et al., 1981, Solter and Knowles 1978), the expression of specific proteins such as OCT-3 (Rosner et al., 1990), or the expression of telomerase activity (Prowse and Greider 1995). The OCT-3, REX-1 and UTF-1 proteins, inter alia, have, to date, only been identified in mice. Ultimate verification of the pluripotent nature is based on analysis of the morphogenetic potentialities of these cells after they have been transplanted into host embryos, which represent a very laborious test.

Another difficulty encountered in culturing ES cells comprises the obtaining of cell populations with a low and satisfactory degree of heterogeneity, and the problem of controlling the growth in culture of nonpluripotent cells. In fact, a particular problem is associated with the continual presence of certain differentiated cell types, that is to say the cells are capable of eliminating the ES cells from the culture by inducing differentiation thereof or programmed cell death thereof.

The present invention proposes to simplify the identification of the pluripotent nature of avian cells in culture, by disclosing a nucleic acid sequence (ens-1 gene) expressed specifically and selectively by the pluripotent cells.

Thus, a subject of the invention is a nucleic acid characterized in that it comprises a nucleic acid sequence chosen from the group of following sequences:

a) SEQ ID No. 1, or the fragment corresponding to nucleotides 1409-2878 of SEQ ID No. 1;

b) the sequence of a fragment of at least 15 consecutive nucleotides of a sequence chosen from SEQ ID No. 1, in particular the fragment corresponding to nucleotides 3111-3670 of SEQ ID No. 1;

c) a nucleic acid sequence having a percentage identity of at least 80%, after the optimal alignment, with a sequence defined in a) or b), said sequence not being defined by nucleotides 2308-2927 or 3094-3753 of SEQ ID No. 1;

d) a nucleic acid sequence which hybridizes, under high stringency conditions, with a nucleic acid sequence defined in a) or b), said sequence not being defined by nucleotides 2308-2927 or 3094-3753 of SEQ ID No. 1;

e) the complementary sequence or the RNA sequence corresponding to a sequence as defined in a), b), c) or d).

Preferably, the base present at 2773 of SEQ ID No. 1 is a “t”, the corresponding codon then encoding a threonine.

The nucleic acid sequence according to the invention defined in c) has a percentage identity of at least 80%, after optimal alignment, with a sequence as defined in a) or b) above, preferably 90%, most preferably 98%. The sequence defined in c), d) or in e) is preferably compared with one of the sequences defined in a).

The terms “nucleic acid”, “nucleic acid sequence”, “polynucleotide”, “oligonucleotide”, “polynucleotide sequence” and “nucleotide sequence”, terms which will be used equally in the present description, are intended to denote a precise string of nucleotides, which may or may not be modified, making it possible to define a fragment or a region of a nucleic acid, which may or may not comprise unnatural nucleotides, and which may correspond equally to a double-stranded DNA, a single-stranded DNA and transcription products of said DNAs. Thus, the nucleic acid sequences according to the invention also encompass PNAs (peptide nucleic acids), or the like.

It should be understood that the present invention does not relate to the nucleotide sequences in their natural chromosomal environment, that is to say in the natural state. They are sequences which have been isolated and/or purified, that is to say they have been taken directly or indirectly, for example by copying, their environment having been at least partially modified. Thus, nucleic acids obtained by chemical synthesis are also intended to be denoted.

For the purpose of the present invention, the term “percentage identity” between two nucleic acid or amino acid sequences is intended to denote a percentage of nucleotides or of amino acid residues which are identical between the two sequences to be compared, obtained after the best alignment, this percentage being purely statistical and the differences between the two sequences being distributed randomly and over their entire length. The term “best alignment” or “optimal alignment” is intended to denote the alignment for which the percentage identity determined as below is highest. Sequence comparisons between two nucleic acid or amino acid sequences are conventionally carried out by comparing these sequences after having optimally aligned them, said comparison being carried out by segment or by “window of comparison” so as to identify and compare local regions of sequence similarity. The optimal alignment of the sequences for the comparison may be carried out, besides manually, by means of the local homology algorithm of Smith and Waterman (1981), by means of the local homology algorithm of Neddleman and Wunsch (1970), by means of the similarity search method of Pearson and Lipman (1988), by means of computer programs using these algorithms (GAP, BESTFIT, BLAST P, BLAST N, FASTA and TFASTA in the Wisconsin Genetics Software Package, Genetics Computer Group, 575 Science Dr., Madison, Wis.). In order to obtain the optimal alignment, the BLAST program is preferably used, with the BLOSUM 62 matrix. The PAM or PAM250 matrices may also be used.

The percentage identity between two nucleic acid or amino acid sequences is determined by comparing these two sequences aligned optimally, the nucleic acid or amino acid sequence to be compared possibly comprising additions or deletions with respect to the reference sequence for optimal alignment between the two sequences. The percentage identity is calculated by determining the number of identical positions for which the nucleotide or the amino acid residue is identical between the two sequences, dividing this number of identical positions by the total number of positions compared and multiplying the result obtained by 100 so as to obtain the percentage identity between these two sequences.

The expression “nucleic acid sequences having a percentage identity of at least 80%, preferably 90%, more preferably 98%, after optimal alignment with a reference sequence” is intended to denote the nucleic acid sequences which, compared with the reference nucleic acid sequence, have certain modifications, such as in particular a deletion, a truncation, an extension, a chimeric fusion and/or a substitution, in particular of the point type, and the nucleic acid sequence of which exhibits at least 80%, preferably 90%, more preferably 98%, identity, after optimal alignment, with the reference nucleic acid sequence. They are preferably sequences whose complementary sequences are capable of hybridizing specifically with the sequence SEQ ID No. 1 of the invention. Preferably, the specific or high stringency hybridization conditions will be such that they ensure at least 80%, preferably 90%, more preferably 98%, identity, after optimal alignment, between one of the two sequences and the sequence complementary to the other.

Hybridization under high stringency conditions means that the conditions of temperature and of ionic strength are chosen such that they allow the hybridization between two complementary DNA fragments to be maintained. By way of illustration, high stringency conditions for the hybridization step for the purpose of defining the polynucleotide fragments described above are advantageously as follows.

The DNA-DNA or DNA-RNA hybridization is carried out in two steps: (1) prehybridization at 42° C. for 3 hours in phosphate buffer (20 mM, pH 7.5) containing 5×SSC (1×SSC corresponds to a solution of 0.15 M NaCl+0.015 M sodium citrate), 50% of formamide, 7% of sodium dodecyl sulfate (SDS), 10× Denhardt's, 5% of dextran sulfate and 1% of salmon sperm DNA; (2) hybridization per se for 20 hours at a temperature which depends on the length of the probe (i.e.: 42° C. for a probe >100 nucleotides in length), followed by 2 washes of 20 minutes at 20° C. in 2×SSC+2% SDS, and 1 wash of 20 minutes at 20° C. in 0.1×SSC+0.1% SDS. The final wash is carried out in 0.1×SSC+0.1% SDS for 30 minutes at 60° C. for a probe >100 nucleotides in length. The high stringency hybridization conditions described above for a polynucleotide of defined length may be adjusted by those skilled in the art for longer or shorter oligonucleotides, according to the teaching of Sambrook et al., 1989.

Among the nucleic acid sequences having a percentage identity of at least 80%, preferably 90%, more preferably 98%, after optimal alignment, with the sequence according to the invention, preference is also given to the nucleic acid sequences which are variants of SEQ ID No. 1, or of fragments thereof, that is to say all the nucleic acid sequences corresponding to allelic variants, that is to say individual variations of the sequence SEQ ID No. 1. These natural mutated sequences correspond to polymorphisms present in birds, in particular in galliform birds. Preferably, the present invention relates to the variant nucleic acid sequences in which the mutations lead to a modification of the amino acid sequence of the polypeptide, or of fragments thereof, encoded by the normal sequence of SEQ ID No. 1.

The expression “variant nucleic acid sequence” is also intended to denote any RNA or cDNA resulting from a mutation and/or variation of a splice site of the genomic nucleic acid sequence the cDNA of which has the sequence SEQ ID No. 1.

The invention preferably relates to a purified or isolated nucleic acid according to the present invention, characterized in that it comprises or consists of the sequence SEQ ID No. 1, the sequence complementary thereto or the RNA sequence corresponding to SEQ ID No. 1.

Preferably, the fragments which hybridize to the nucleic acid according to the invention, or which are homologous to said nucleic acid, are not defined by nucleotides 2308-2927 or 3094-3753 of SEQ ID No. 1, which correspond approximately to ESTs (GenBank numbers AJ397754 and AJ393785) which have been obtained by systematic sequencing and with regard to which no piece of data, in particular functional data, has been provided. For this reason, these disclosures should be considered to be accidental disclosures.

The probes or primers, characterized in that they comprise a sequence of a nucleic acid according to the invention, are also part of the invention.

Thus, the present invention also relates to the primers or the probes according to the invention which may make it possible in particular to demonstrate or to distinguish the variant nucleic acid sequences, or to identify the genomic sequence of the gene the cDNA of which is represented by SEQ ID No. 1, in particular using an amplification method such as the PCR method, or a related method.

The invention also relates to the use of a nucleic acid sequence according to the invention, as a probe or primer, for detecting, identifying, assaying and/or amplifying nucleic acid sequences.

The invention also relates to the use of a nucleic acid sequence according to the invention as a sense or antisense oligonucleotide.

According to the invention, the polynucleotides which can be used as a probe or as a primer in methods for detecting, identifying, assaying or amplifying a nucleic acid sequence are a minimum of 15 bases, preferably 20 bases, or better still 25 to 30 bases, in length.

The probes and primers according to the invention may be labeled directly or indirectly with a radioactive or nonradioactive compound using methods well known to those skilled in the art, in order to obtain a detectable and/or quantifiable signal.

The polynucleotide sequences according to the invention which are unlabeled can be used directly as a probe or primer.

The sequences are generally labeled so as to obtain sequences which can be used for many applications. The primers or the probes according to the invention are labeled with radioactive elements or with nonradioactive molecules.

Among the radioactive isotopes used, mention may be made of ³²P, ³³P, ³⁵S, ³H or ¹²⁵I. The nonradioactive entities are selected from ligands such as biotins, avidins, streptavidins, or dioxygenin, haptens, dyes and luminescent agents, such as radioluminescent, chemiluminescent, bioluminescent, fluorescent or phosphorescent agents.

The polynucleotides according to the invention may thus be used as a primer and/or probe in methods using in particular the PCR (polymerase chain reaction) technique (Rolfs et al., 1991). This technique requires choosing pairs of oligonucleotide primers bordering the fragment which must be amplified. Reference may, for example, be made to the technique described in U.S. Pat. No. 4,683,202. The amplified fragments can be identified, for example after agarose or polyacrylamide gel electrophoresis, or after a chromatographic technique such as gel filtration or ion exchange chromatography, and then sequenced. The specificity of the amplification can be controlled using, as primers, the nucleotide sequences of polynucleotides of the invention and, as matrices, plasmids containing these sequences or else the derived amplification products. The amplified nucleotide fragments may be used as reagents in hybridization reactions in order to demonstrate the presence, in a biological sample, of a target nucleic acid of sequence complementary to that of said amplified nucleotide fragments.

The invention is also directed toward the nucleic acids which can be obtained by amplification using primers according to the invention.

Other techniques for amplifying the target nucleic acid may advantageously be employed as an alternative to PCR (PCR-like) using a pair of primers of nucleotide sequences according to the invention. The term “PCR-like” is intended to denote all the methods using direct or indirect reproductions of nucleic acid sequences, or else in which the labeling systems have been amplified; these techniques are, of course, known. In general, they involve amplifying the DNA with a polymerase; when the sample of origin is an RNA, a reverse transcription should be carried out beforehand. A large number of methods currently exists for this amplification, such as, for example, the SDA (strand displacement amplification) technique (Walker et al., 1992), the TAS (transcription-based amplification system) technique described by Kwoh et al. (1989), the 3SR (self-sustained sequence replication) technique described by Guatelli et al. (1990), the NASBA (nucleic acid sequence based amplification) technique described by Kievitis et al. (1991), the TMA (transcription mediated amplification) technique, the LCR (ligase chain reaction) technique described by Landegren et al. (1988), the RCR (repair chain reaction) technique described by Segev (1992), the CPR (cycling probe reaction) technique described by Duck et al. (1990), and the Q-beta-replicase amplification technique described by Miele et al. (1983). Some of these techniques have since been improved.

When the target polynucleotide to be detected is an mRNA, an enzyme of the reverse transcriptase type is advantageously used, prior to carrying out an amplification reaction using the primers according to the invention or to carrying out a method of detection using the probes of the invention, in order to obtain a cDNA from the mRNA contained in the biological sample. The cDNA obtained will then serve as a target for the primers or the probes used in the amplification or detection method according to the invention.

The probe hybridization technique may be carried out in various ways (Matthews et al., 1988). The most general method consists in immobilizing the nucleic acid extracted from the cells of various tissues or from cells in culture, on a support (such as nitrocellulose, nylon or polystyrene), and in incubating the immobilized target nucleic acid with the probe, under well-defined conditions. After hybridization, the excess probe is removed and the hybrid molecules formed are detected using the appropriate method (measuring the radioactivity, the fluorescence or the enzymatic activity linked to the probe).

According to another embodiment of the nucleic acid probes according to the invention, the latter may be used as capture probes. In this case, a probe, termed “capture probe”, is immobilized on a support and is used to capture, by specific hybridization, the target nucleic acid obtained from the biological sample to be tested, and the target nucleic acid is then depicted using a second probe, termed “detection probe”, labeled with a readily detectable element.

Among the advantageous nucleic acid fragments, mention should thus be made in particular of antisense oligonucleotides, i.e. oligonucleotides the structure of which ensures, by hybridization with the target sequence, inhibition of expression of the corresponding product. Mention should also be made of sense oligonucleotides which, by interacting with proteins involved in regulating the expression of the corresponding protein, will induce either inhibition or activation of this expression.

In a particular embodiment of the invention, the nucleic acid according to the invention encodes a polypeptide which has a continuous fragment of at least 200 amino acids of the protein SEQ ID No. 2, preferably 300 amino acids, and most preferably encodes the protein SEQ ID No. 2. This polypeptide is also a subject of the invention.

In fact, the present invention also relates to an isolated polypeptide, characterized in that it comprises a polypeptide chosen from:

a) a polypeptide of sequence SEQ ID No. 2;

b) a variant polypeptide of a polypeptide of sequence SEQ ID No. 2;

c) a polypeptide homologous to a polypeptide defined in a) or b), comprising at least 80% identity with said polypeptide of a);

d) a fragment of at least 15 consecutive amino acids of a polypeptide defined in a), b) or c);

e) a biologically active fragment of a polypeptide defined in a), b) or c).

Preferably, the amino acid at position 455 is a threonine.

For the purpose of the present invention, the term “polypeptide” is intended to denote proteins or peptides.

The expression “biologically active fragment” is intended to mean a fragment having the same biological activity as the peptide fragment from which it is deduced, preferably within the same order of magnitude (to within a factor of 10). A biologically active fragment of the ENS-1 protein therefore consists of a polypeptide derived from SEQ ID No. 2 which may also have a role in the characteristic of pluripotencey of ES cells.

Preferably, a polypeptide according to the invention is a polypeptide consisting of the sequence SEQ ID No. 2 (corresponding to the protein encoded by the ens-1 gene) or of a sequence having at least 80% identity with SEQ ID No. 2 after optimal alignment.

The sequence of the polypeptide has a percentage identity of at least 80%, after optimal alignment, with the sequence SEQ ID No. 2, preferably 90%, more preferably 98%.

The expression “polypeptide the amino acid sequence of which has a percentage identity of at least 80%, preferably 90%, more preferably 98%, after optimal alignment, with a reference sequence” is intended to denote the polypeptides having certain modifications compared to the reference polypeptide, such as in particular one or more deletions and/or truncations, an extension, a chimeric fusion and/or one or more substitutions.

Among the polypeptides the amino acid sequence of which has a percentage identity of at least 80%, preferably 90%, more preferably 98%, after optimal alignment, with the sequence SEQ ID No. 2 or with a fragment thereof according to the invention, preference is given to the variant polypeptides encoded by the variant nucleic acid sequences as defined previously, in particular the polypeptides the amino acid sequence of which has at least one mutation corresponding in particular to a truncation, deletion, substitution and/or addition of at least one amino acid residue compared with the sequence SEQ ID No. 2 or with a fragment thereof, more preferably the variant polypeptides having a mutation associated with a loss of pluripotent nature of the cells containing them.

The present invention also relates to the cloning and/or expression vectors comprising a nucleic acid or encoding a polypeptide according to the invention. Such a vector may also contain the elements required for the expression and, optionally, the secretion of the polypeptide in a host cell. Such a host cell is also a subject of the invention.

The vectors characterized in that they comprise a promoter and/or regulator sequence according to the invention are also part of the invention.

Said vectors preferably comprise a promoter, translation initiation and termination signals, and also regions suitable for regulating transcription. It must be possible for them to be maintained stably in the cell and they may optionally contain particular signals specifying secretion of the translated protein.

These various control signals are chosen as a function of the cellular host used. To this effect, the nucleic acid sequences according to the invention can be inserted into vectors which replicate autonomously in the chosen host, or vectors which integrate in the chosen host.

Among the systems which replicate autonomously, use is preferably made, depending on the host cell, of systems of the plasmid or viral type, the viral vectors possibly being in particular adenoviruses (Perricaudet et al., 1992), retroviruses, lentiviruses, poxviruses or herpesviruses (Epstein et al., 1992). Those skilled in the art are aware of the technology which can be used for each of these systems.

When integration of the sequence into the chromosomes of the host cell is desired, use may be made, for example, of systems of the plasmid or viral type; such viruses are, for example, retroviruses (Temin, 1986) or AAVs (Carter, 1993).

Among the nonviral vectors, preference is given to naked polynucleotides such as naked DNA or naked RNA according to the technology developed by the company VICAL, bacterial artificial chromosomes (BACs), yeast artificial chromosomes (YACs) for expression in yeast, mouse artificial chromosomes (MACs) for expression in murine cells and, preferably, human artificial chromosomes (HACs) for expression in human cells.

In avian cells, retroviruses, avian adenoviruses, poxviruses or else DNA introduced by transfection or electroporation may be used as an expression vector.

Such vectors are prepared according to the methods commonly used by those skilled in the art, and the clones resulting therefrom can be introduced into a suitable host using standard methods, such as, for example, lipofection, electroporation, heat shock, transformation after chemical permeabilization of the membrane, or cell fusion.

The invention also comprises the host cells, in particular the eukaryotic and prokaryotic cells, transformed with the vectors according to the invention, and also the transgenic animals, preferably the birds or mammals, except humans, comprising one of said transformed cells according to the invention. In particular, the invention comprises the animals comprising the ens-1 gene having genetic markers inserted into this gene.

Among the cells which can be used for the purpose of the present invention, mention may be made of bacterial cells (Olins and Lee, 1993), but also yeast cells (Buckholz, 1993), and also animal cells, in particular mammalian cell cultures (Edwards and Aruffo, 1993), and especially Chinese hamster ovary (CHO) cells. Mention may also be made of insect cells in which it is possible to use methods employing, for example, baculoviruses (Luckow, 1993). A preferred cellular host for expressing the proteins of the invention consists of COS cells.

Among the avian cells which can be used, mention may be made of LMH chicken hematoma cells, QT6 immortalized quail cells, and primary or immortalized chicken, quail or duck fibroblasts.

The invention also relates to a host cell containing a nucleic acid according to the invention, characterized in that it is an avian ES cell also modified by introducing an exogenous gene, said exogenous gene being expressed only and specifically when said cell is maintained in the pluripotent state. Preferably, said exogenous gene is a reporter gene chosen from lacZ, GFP, luciferase, ROSA-β-geo, and a gene for resistance to an antibiotic, in particular the genes for resistance to neomycin, hygromycin, phleomycin or puromycin).

These cells according to the invention are very useful for screening for compounds which make it possible to induce differentiation of the pluripotent cells, or for medium for culturing cells while at the same time maintaining their pluripotent nature.

Another host cell of interest according to the invention consists of an avian cell containing a nucleic acid according to the invention, also modified by introducing an exogenous nucleic acid, said exogenous nucleic acid being integrated into said nucleic acid according to the invention. According to a preferred embodiment of the invention, said exogenous nucleic acid is a gene of therapeutic interest, optionally preceded by a spatio-temporal promoter and/or by terminator sequences. In another embodiment, said exogenous nucleic acid is a genetic marker which may be chosen from lacZ, GFP, alkaline phosphatase, thymidine kinase, and genes for resistance to antibiotics. (Among which are neomycin, hygromycin, phleomycin and puromycin).

Preferably, the avian host cells described above are characterized in that the bird belongs to the order Galliformes, and is in particular a chicken or a quail.

In this case, said reporter gene is integrated under the control of the promoter of the ens-1 gene and/or said exogenous nucleic acid (gene of therapeutic interest and/or genetic marker) is integrated into the ens-1 gene.

It is thus possible to use the promoter identified in the present application, corresponding to nucleotides 3111-3670 of SEQ ID No. 1. It is also possible to modify this promoter, by reducing the number of nucleotides, or by introducing additional ones, or even by performing mutations on certain nucleotides. Those skilled in the art are aware of the protocols for carrying out said modifications, and also for testing the promoter thus obtained for expression in pluripotent stem cells. It has thus been shown in particular that it is possible to insert a guanine at position 3654 of SEQ ID No. 1 without losing the promoter activity of the fragment thus modified.

Thus, the invention also relates to the use of a nucleic acid corresponding to nucleotides 3111-3670 of SEQ ID No. 1 as a promoter of a gene of interest for specific expression of said gene of interest in avian pluripotent cells. A gene of interest is either a marker gene (luciferase, GFP, β-galactosidase, etc.) or it can be a gene encoding a protein such as a growth factor, a cytokine, a protein involved in immune recognition, a protein with therapeutic value, etc. It is interesting to note that the “TATA box” has also been identified, at nucleotides 3645-3651 of SEQ ID No. 1, and it is a subject of the invention.

A preferred cell according to the invention is a 9N2.5 cell, deposited with the Collection Nationale de Culture des Microorganismes [National Collection of Cultures and Microorganisms], on May 11, 2000, under the identification number I-2477.

The cells according to the invention are preferably pluripotent ES cells, but it should be understood that the invention also relates to the differentiated avian cells which derive from an ES cell according to the invention. These cells can in particular be differentiated using retinoic acid, according to the teachings of patent application WO 96/12793.

The invention also relates to the transgenic animals which contain a cell according to the invention. Among the animals according to the invention preference is given to birds, in particular the members of the order Galliformes. These transgenic birds will be particularly advantageous for studying modifications in the ens-1 gene or in its promoter.

It is also possible to introduce a nucleic acid according to the invention into birds and other animals, such as rodents, in particular mice, rats or rabbits, in order to express a polypeptide according to the invention.

These transgenic animals are obtained, for example, by homologous recombination on embryonic stem cells, transfer of these stem cells to embryos, selection of the chimeras affected in the reproductive line, and growth of said chimeras. They may also be obtained by microinjection of naked DNA into the fertilized oocyte.

The transgenic animals according to the invention can thus overexpress the gene encoded in the protein according to the invention, or their homologous gene, or express said gene into which a mutation is introduced, or else express a transgene comprising portions of the ens-1 gene associated with coding sequences intended to produce a protein.

Alternatively, the transgenic birds according to the invention can be made deficient for the gene encoding the polypeptide of sequence SEQ ID No. 2, or a homologous gene, by inactivation using the LOXP/CRE recombinase system (Rohlmann et al., 1996) or any other system for inactivating the expression of this gene.

The invention also relates to the use of a nucleic acid sequence according to the invention, for synthesizing recombinant polypeptides.

The method for producing a polypeptide of the invention in recombinant form, which is itself included in the present invention, is characterized in that the transformed cells, in particular the cells or mammals of the present invention, are cultured under conditions which allow the expression of a recombinant polypeptide encoded by nucleic acid sequence according to the invention, and in that said recombinant polypeptide is recovered.

The recombinant polypeptides, characterized in that they can be obtained using said method of production, are also part of the invention.

The recombinant polypeptides obtained as indicated above can be in both glycosylated and nonglycosylated form, and may or may not have the natural tertiary structure.

The sequences of the recombinant polypeptides may also be modified in order to improve their solubility, in particular in aqueous solvents.

Such modifications are known to those skilled in the art, such as, for example, deletion of hydrophobic domains or substitution of hydrophobic amino acids with hydrophilic amino acids.

These polypeptides may be produced using the nucleic acid sequences defined above, according to the techniques for producing recombinant polypeptides known to those skilled in the art. In this case, the nucleic acid sequence used is placed under the control of signals which allow its expression in a cellular host.

An effective system for producing a recombinant polypeptide requires having a vector and a host cell according to the invention.

These cells can be obtained by introducing into host cells a nucleotide sequence inserted into a vector as defined above, and then culturing said cells under conditions which allow the replication and/or expression of the transfected nucleotide sequence.

The methods used for purifying a recombinant polypeptide are known to those skilled in the art. The recombinant polypeptide may be purified from cell lysates and extracts or from the culture medium supernatant, by methods used individually or in combination, such as fractionation, chromatography methods, immunoaffinity techniques using specific monoclonal or polyclonal antibodies, etc.

The polypeptides according to the present invention can also be obtained by chemical synthesis using one of the many known forms of peptide synthesis, for example techniques using solid phases (see in particular Stewart et al., 1984) or techniques using partial solid phases, by fragment condensation or by conventional synthesis in solution.

The polypeptides obtained by chemical synthesis and which may comprise corresponding unnatural amino acids are also included in the invention.

The mono- or polyclonal antibodies, or fragments thereof, chimeric antibodies or immunoconjugates, characterized in that they are capable of specifically recognizing a polypeptide according to the invention, are part of the invention.

Specific polyclonal antibodies may be obtained from a serum of an animal immunized against polypeptides according to the invention, in particular produced by genetic recombination or by peptide synthesis, according to the usual procedures.

The advantage of antibodies which specifically recognize certain polypeptides, variants or immunogenic fragments thereof according to the invention is in particular noted.

The mono- or polyclonal antibodies, or fragments thereof, chimeric antibodies or immunoconjugates characterized in that they are capable of specifically recognizing the polypeptide of sequence SEQ ID No. 2 are particularly preferred.

The specific monoclonal antibodies may be obtained according to the conventional method of hybridoma culture described by Kohler and Milstein (1975).

The antibodies according to the invention are, for example, chimeric antibodies, humanized antibodies, or Fab or F(ab′)₂ fragments. They may also be in the form of immunoconjugates or of labeled antibodies, in order to obtain a detectable and/or quantifiable signal.

The invention also relates to methods for detecting and/or purifying a polypeptide according to the invention, characterized in that they use an antibody according to the invention.

The invention also comprises purified polypeptides, characterized in that they are obtained using a method according to the invention.

Moreover, besides their use for purifying polypeptides, the antibodies of the invention, in particular the monoclonal antibodies, may also be used for detecting these polypeptides in a biological sample.

They thus constitute a mean for the immunocytochemical or immunohistochemical analysis of the expression of the polypeptides according to the invention, in particular the polypeptide of sequence SEQ ID No. 2, or a variant thereof, on specific tissue sections, for example using immunofluorescence, gold labeling and/or enzymatic immunoconjugates.

They may in particular make it possible to demonstrate the expression of these polypeptides in the tissues or biological specimens.

More generally, the antibodies of the invention may advantageously be used in any circumstances where the expression of a polypeptide according to the invention, normal or mutated, must be observed.

Thus, a method for detecting a polypeptide according to the invention, in a biological sample, comprising the steps of bringing the biological sample into contact with an antibody according to the invention and demonstrating the antigen-antibody complex formed, is also a subject of the invention, as is a kit for carrying out such a method. Such a kit in particular contains:

a) a monoclonal or polyclonal antibody according to the invention;

b) optionally, reagents for constituting a medium suitable for the immunoreaction;

c) the reagents for detecting the antigen-antibody complex produced during the immunoreaction.

These antibodies may be obtained directly from human serum, or may be obtained from animals immunized with polypeptides according to the invention, and then “humanized”.

The antibodies according to the invention are very useful for determining the presence of the polypeptide SEQ ID No. 2, and thus make it possible to determine the pluripotent nature of an avian ES cell.

A method for determining the pluripotent nature of an avian ES cell, characterized in that a product of expression of the gene corresponding to SEQ ID No. 1 or of the mRNA of SEQ ID No. 1 is determined, is also a subject of the invention.

The invention in fact discloses the sequence of the ens-1 gene, which is specifically expressed in avian ES cells, in particular ES cells of galliforms, when these cells are pluripotent. The methods for detecting expression of a gene, applied to this gene, therefore make it possible to rapidly determine the nature of the cells studied.

In particular, as described above, the product of expression of the gene can be detected, using, for example, antibodies according to the invention, by Western blotting or other methods described previously.

It is also possible to detect the mRNA of SEQ ID No. 1 by Northern blotting or by RT-PCR using a probe or primers according to the invention.

Detection of the expression of this gene can also be carried out using a DNA chip or a protein chip, which contain, respectively, a nucleic acid or a polypeptide according to the invention. Such chips are also subjects of the invention.

A protein chip according to the invention also makes it possible to study the interactions between the polypeptides according to the invention and other proteins or chemical compounds, and may thus be useful for screening for compounds which interact with the polypeptides according to the invention.

The applicant has shown that the ens-1 gene is found only in birds of the galliform family. Thus, the invention also relates to a method for classifying a bird as belonging to the order Galliformes, characterized in that the presence of a nucleic acid according to the invention, in particular the presence of SEQ ID No. 1, is detected in the gene of said bird.

This property that the ens-1 gene is found only in galliform birds makes it possible to define a method for determining the presence of a sample originating from a bird of the order Galliformes in a food sample, characterized in that the presence of a nucleic acid according to the invention, in particular the presence of SEQ ID No. 1, is detected in said sample.

The presence of a nucleic acid according to the invention in a biological or food sample, or in the genome of a bird, can be detected in various ways. In particular, it is possible to define a method for detecting and/or assaying a nucleic acid according to the invention in a biological or food sample, characterized in that it comprises the following steps:

a) bringing said sample into contact with a polynucleotide as claimed in one of claims 1 to 3, which is labeled;

b) detecting and/or assaying the hybrid formed between said polynucleotide and the nucleic acid of said sample.

It is also possible to detect and/or assay a nucleic acid according to the invention in a biological or food sample by carrying out a step of amplification of the nucleic acids of said sample using primers chosen from nucleic acids according to the invention.

As demonstrated in the examples, the nucleic acid according to the invention is expressed in avian ES cells only when the cells are pluripotent in nature. Moreover, the ES cells modified according to the invention, with a reporter gene expressed specifically when they are pluripotent, and in particular the 9N2.5 cells, can be used to screen for compounds of interest.

In particular, they may be used in a method for screening for a substance or for a medium capable of inducing differentiation of pluripotent cells, characterized in that it comprises the following steps:

a) maintaining ES cells according to the invention in a culture medium making it possible to maintain the pluripotent phenotype;

b) adding said substance to said culture medium or replacing said culture medium with the medium to be tested;

c) determining the induction of differentiation by the absence of expression of the protein SEQ ID No. 2 or of the exogenous gene.

This method is preferably carried out with ES cells modified by inserting a reporter gene under the control of the promoter of the ens-1 gene, and the absence of expression of said reporter gene is detected. Use is preferably made of 9N2.5 cells, and the absence of expression of β-galactosidase is detected.

It is also possible to use the cells according to the invention to screen for substances capable of restoring the pluripotent nature of differentiated cells, using a method comprising the following steps:

a) maintaining differentiated cells in a suitable culture medium;

b) replacing said culture medium with a medium which makes it possible to maintain a pluripotent phenotype and which contains said substance to be tested;

c) determining the restoration of the pluripotent nature of said cells by the expression of the protein SEQ ID No. 2 or of the exogenous gene, in said cells.

This method is again advantageously used with differentiated cells according to the invention, modified by inserting a reporter gene into the ens-1 gene or under the control of its promoter. Differentiated 9N2.5 cells, which allow detection of β-galactosidase expression, are advantageously used.

The methods described above are also subjects of the invention, as are the media or substances obtained using said methods.

Such a substance according to the invention may be a compound having a chemical structure (of the small organic molecule type), a lipid, a sugar, a protein, a peptide, a protein-lipid, protein-sugar, peptide-lipid or peptide-sugar hybrid compound, or a protein or peptide to which chemical branching has been added.

Among the chemical compounds envisaged, they may contain one or more rings, which may or may not be aromatic, and also several residues of any kind (in particular lower alkyl, i.e. having between 1 and 6 carbon atoms).

It is extremely important to determine the genes involved in the characteristic of pluripotency of ES cells, or to benefit from having a marker for said characteristic. In fact, due to the capacity of these cells to contribute to the morphagenesis of all tissues, a genetic modification of these cells makes it possible to ensure that the characteristics sought will be found in all the tissues of the animal formed. Moreover, the introduction of exogenous genes at the locus of the ens-1 gene, under the control of varying promoters with spatio-temporal specificity, may make it possible to obtain transgenic animals expressing said genes in given tissues or at given developmental stages. In fact, since the specificity of the ens-1 gene is that it is expressed only if the host cell is pluripotent in nature, the introduction of an exogenous nucleic acid into this locus should not impair the development of the embryo.

It is therefore possible to introduce genes of therapeutic interest, for example encoding therapeutic proteins (hormones, growth factors, lymphokines), so as to be able to produce these proteins during the development of the embryo. It may in fact be very advantageous to produce therapeutic proteins in the eggs, the shell of which ensures a sterile environment.

It is also possible to use pluripotent cells according to the invention in order to make them colonize the germinal tissue of animals, in particular of birds, more preferably of the order Galliformes, so that particular genetic characteristics may be transmitted to their progeny. This makes it possible to improve industrial races of chickens, turkeys, quails and the like, in a manner which is particularly advantageous in economic terms.

It is also possible to use the compounds chosen from

a) a nucleic acid according to the invention;

b) a polypeptide according to the invention;

c) a vector according to the invention;

d) a cell according to the invention;

e) an antibody according to the invention;

f) a substance according to the invention,

as a medicinal product, in order, as appropriate, to allow restoration of the pluripotent nature of avian cells or, on the other hand, to induce differentiation of ES cells.

The present invention therefore opens up the pathway to a better characterization of the pluripotent nature of ES cells by providing the sequence of a marker for these cells. It remains, however, to be determined whether this gene is a factor essential to this nature. Thus, introducing the ens-1 gene into differentiated cells, for example of a plasmid under the control of a suitable promoter, and studying the possible restoration of the pluripotent nature of the cells, will make it possible to answer this question. Among suitable promoters, an inducible promoter, for example a promoter inducible with a sugar, will be chosen and the pluripotent nature of the cells when induction of expression of the gene on the plasmid is stopped will be determined. It is also possible to construct a plasmid which leads to excision of the ens-1 gene after a certain amount of time (for example by placing it between two loxP sequences, and introducing a second plasmid encoding the Cre recombinase). To determine the pluripotent nature of cells, it may be advantageous to use the 9N2.5 cells according to the invention, and to search for expression of β-galactosidase after introduction of the plasmid encoding ens-1.

If it is possible to determine that the ens-1 gene is an inducer of the pluripotent nature of cells, a method for restoring said nature (also a subject of the invention), characterized in that the ens-1 gene is expressed in differentiated cells, may be carried out. The methods described above may be used, introducing therein certain improvements known to those skilled in the art.

The examples below make it possible to illustrate the invention and should not be considered as limiting the invention.

### DESCRIPTION OF THE FIGURES

FIG. 1: structure of the \dot{v}ector ROSA-β-geo used to transform the ES cells.

FIG. 2: analysis of the expression of the ROSA-β-geo transcript by RT-PCR in 9N2.5 cells at the time of induction or of differentiation with retinoic acid (+RA), DMSO (+DMSO) or both simultaneously (+RA+DMSO). The control medium contains no inducing factor.

FIG. 3: analysis of the expression of the ROSA-β-geo transcript by Northern blotting at the time of induction of differentiation with retinoic acid. The blot is hybridized with a LacZ probe.

FIG. 4: analysis of the expression of the ROSA-β-geo transgene by revealing β-galactosidase activity in embryos which are chimeric for 9N2.5 cells.

FIG. 5: PCR analysis of the presence of the ROSA-β-geo transgene in the chimeric embryos. DNA was extracted either from 9N2.5 cells, or from a 48-hour-old or 4-day-old chimeric embryo resulting from the transplantation of 9N2.5 cells, or from a 48-hour-old or 4-day-old control embryo.

FIG. 6: detection by Southern blotting of the presence of the ROSA-β-geo transgene in the genomic DNA of 9N2.5 cells after digestion with EcoRI (E) or DraI (D).

FIG. 7: detection by Northern blotting of the presence of a transcript comprising the ROSA-β-geo transgene, by hybridization with a LacZ probe.

FIG. 8: A. Northern blotting analysis of the expression of the ens-1 gene in normal chicken ES cells and in 9N2.5 cells, after hybridization with the probes C1, S1 and S2. B. Structure of the complementary DNA of the ens-1 gene (RS=repeat sequences, ORF=open reading frame). The arrows represent the probes C1, S1 and S2 used for the hybridization.

FIG. 9: Northern blotting analysis of the expression of the ens-1 transcripts in normal chicken embryonic stem cells, in 9N2.5 cells, in the chicken embryo at various development stages and in various chick organs. The polyA+RNAs isolated from the total RNAs were hybridized on the blots with the probes C1 or S1, or with a control GAPDH probe.

FIG. 10: analysis of the expression of the ens-1 transcript in the chicken embryo by in situ hybridization.

FIG. 11: PCR amplification carried out on the genomic DNA of various avian species with the ens1 primer S1 (SEQ ID No. 14) and ens1 primer AS1 (SEQ ID No. 15).

FIG. 12: diagram of the organization of the retroviral LTRs, of the expected organization for the ens-1 gene, and of the two constructs used to identify the promoter.

FIG. 13: Activity of the promoters in various cell lines (S: sense promoter, AS: antisense promoter).

FIG. 14: activity of promoter 2 (FIG. 12) during differentiation of ES cells.

### EXAMPLES

### Example 1

### Construction of a Chicken ES Cell Containing a Genetic Marker for Pluripotency

In order to identify a gene specifically expressed in pluripotent ES cells, the “gene trap” strategy was followed. This strategy consists in introducing, into the genome of ES cells, a marker gene which comprises an exogenous coding sequence but which lacks its own promoter. The random insertion of this marker into the genome of the cell will, in certain cases, lead to this exogenous gene being placed downstream of a promoter belonging to the cellular genome. In this configuration, the exogenous gene adopts a regulation of expression very similar if not identical to that of the gene into which it is inserted. Following the expression of the marker gene in the cells thus modified then provides information regarding the pattern of expression of the cellular gene thus “marked”.

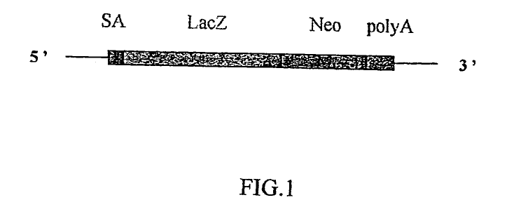

As gene trap system, the inventors used that which exploits the properties of the vector ROSA-β-geo described by Friedrich and Soriano (1991). This system consists of a plasmid which carries the two genes LacZ and Neo^{R}, respectively, fused to one another in the 5′-3′ order. The gene fusion encodes a single LacZ-Neo protein which confers on the cells which produce it both resistance to G418 and β-galactosidase activity. The structure of the plasmid is given in FIG. 1. This plasmid was cleaved with the DraI enzyme, which induces linearization thereof. The linearized plasmid was introduced into chicken ES cells by the electroporation technique. For this, a culture of chicken ES cells maintained under the conditions described in Pain et al. (1996) was used. The ES cells were recovered from the culture dishes by controlled treatment with pronase. The cells in suspension were washed and suspended in Glasgow medium at a concentration of 5×10⁶ in 0.8 ml. Ten micrograms of linearized plasmid were added to the cell suspension, which was kept at 4° C. for 10 minutes. The suspension was then subjected to electroporation treatment consisting of 2 electrical stimulations under the following conditions: 280 V, 500 mF in a 1 mm-thick cuvette in a BioRad electroporator device. The cells were then kept at 4° C. for 10 minutes before being seeded in culture according to the method described in Pain et al. (1996), incorporated by way of reference. Thirty-six hours later, G418 was added to the cultures, at a concentration of 250 μg/ml. The culture medium containing G418 was then changed everyday for 4 days, and then every two days. G418-resistant ES cell clones became apparent after the sixth day. They were sampled individually between 8 and 10 days after the beginning of the culture. These clones were seeded individually in fresh culture medium containing G418 in order to be amplified. They were then stored in liquid nitrogen.

In the electroporated cells, the expression of the ROSA-β-geo marker was analyzed by identifying β-galactosidase activity in situ, according to the following method. The cells in suspension were fixed at 4° C. for a period of 30 minutes in a mixture based on PBS containing 1% of formaldehyde, 0.2% of glutaraldehyde and 0.02% of Nonidet P-40. The cells were then incubated at 37° C. for a period which could range from 1 to 24 hours, in PBS containing 1 mg/ml of 5-bromo-4-chloro-3-indolyl β-D-galactopuranoside, 5 mM of K₃Fe(CN)₆, 5 mM of K₄Fe(CN)₆, 2 mM of MgCl₂ and 0.02% of Nonidet P-40. The cells expressed in the β-galactosidase marker were colored blue.

The aim was to identify ES cells in which the vector ROSA-β-geo was inserted downstream of a promoter which would only function in the ES cells when they were pluripotent. After characterization of several clones, one clone, called 9N2.5, was selected, which gave a positive reaction to the β-galactosidase assay only when the cells were maintained under culture conditions ensuring the persistence of the pluripotent nature of the cells, as described in Pain et al. (1996). The positivity of the test was lost when the 9N2.5 cells were induced into differentiation (see below).

The 9N2.5 clone was amplified in culture in vitro, and then stored in viable form by freezing in liquid nitrogen.

### Example 2

### Characterization of the 9N2.5 Cell

The 9N2.5 cells were maintained under the culture conditions described by Pain et al. (1996), for chicken ES cells. Under these conditions, it was verified that the 9N2.5 cells exhibited the morphology, the telomerase activity and the antigenic epitopes characteristic of chicken ES cells, as described by Pain et al. The cells are also capable of forming embryoid bodies, like the parenteral cells. The electroporation, the selection in G418 and the subsequent amplification of the cells had not therefore impaired their ES cell characteristics.

In order to analyze the expression of the ROSA-β-geo marker in differentiated cells, the 9N2.5 cells were induced into differentiation according to the methods described in Pain et al. These cells were cultured in the absence feeder cells, in the absence of LIF and of cytokines, and in the presence either of retinoic acid at a concentration of 5×10 M or of DMSO at a concentration of 1%. In some cultures, the retinoic acid and the DMSO were added simultaneously. In the ES cell differentiation-inducing media, it was possible to observe the appearance of differentiated cells identical to those which were initially described by Pain et al. (1996) under the same conditions.

After 4 days of culture in the differentiation media, the cells became completely negative for the β-galactosidase activity assay. In order to confirm the lack of expression of the ROSA-β-geo transgene, its expression was followed, mainly by searching for LacZ mRNAs by the RT-PCR technique. For this, the primers SEQ ID No. 3 and SEQ ID No. 4 were used:

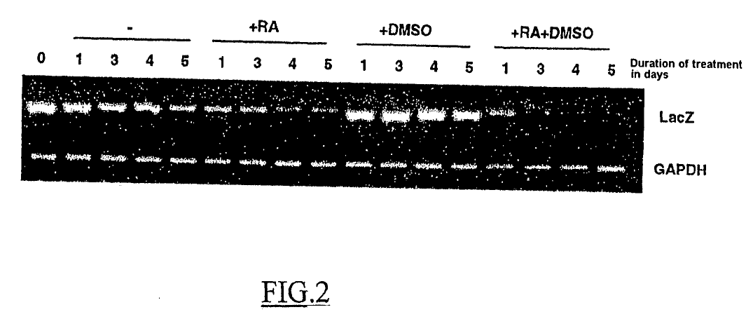

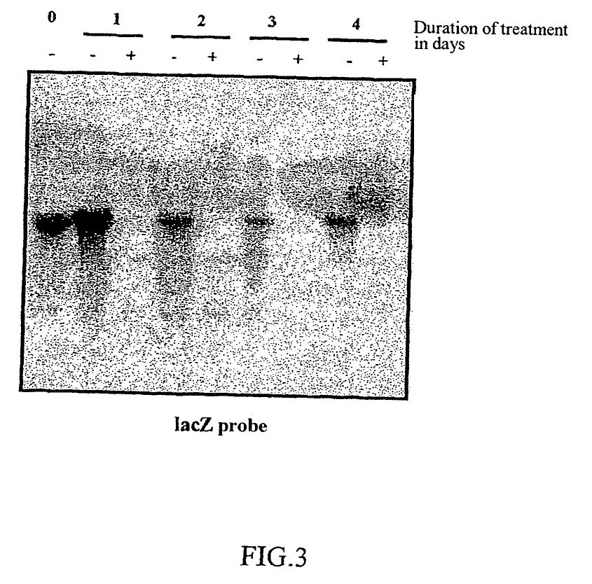

As shown in FIG. 2, the amount of RNA produced by the ROSA-β-geo transgene does not change during 5 days of culturing the cells in the culture medium which maintains pluripotency (ES medium). On the other hand, in the differentiation culture media containing either retinoic acid alone, or DMSO, or retinoic acid and DMSO, the amount of ROSA-β-geo mRNA decreased greatly after 4 days of culturing. For confirmation, the ROSA-β-geo mRNAs were also analyzed by the Northern blotting technique, using a labeled probe specific for the LacZ sequence. As shown in FIG. 3, in the presence of retinoic acid, the LacZ mRNAs became virtually undetectable after two days of culturing, whereas their expression was maintained in the culture medium lacking retinoic acid.

Conclusion

The 9N2.5 cells selected expressed the ROSA-β-geo transgene when they are maintained in the pluripotent state. Expression of the transgene ceases very rapidly after induction of differentiation of these cells in culture.

### Example 3

### Assay for Expression of the ROSA-β-geo Transgene in the 9N2.5 Cells In Vivo

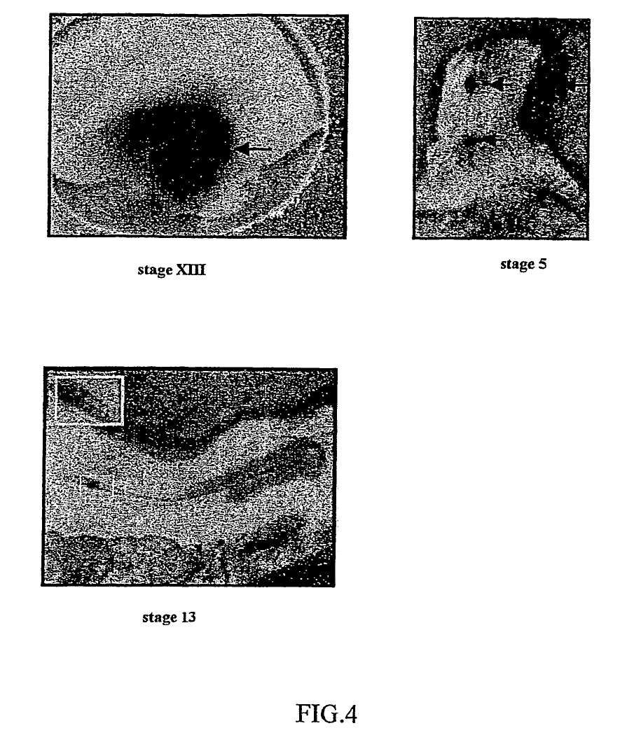

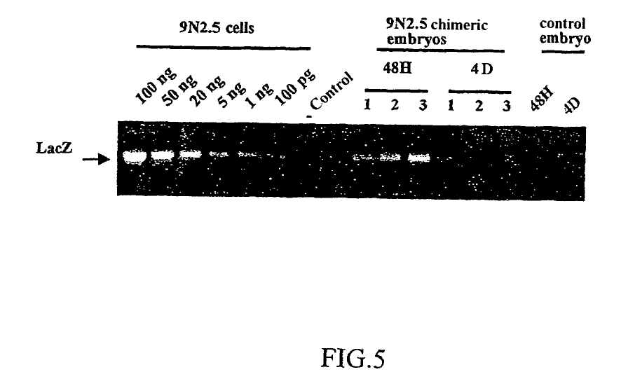

In order to analyze the developmental potentiality of the 9N2.5 cells and the expression of the ROSA-β-geo transgene in an embryo in vivo, the 9N2.5 cells were transplanted into chicken embryos at stage X according to the Eyal-Giladi and Kochav scale (1976) (E-G & K scale), according to the protocol described by Pain et al. (1996). The presence of the descendants of the injected cells was sought in the embryos at various developmental stages after transplantation, using the β-galactosidase assay. As is shown in FIG. 4, aggregates of cells positive for β-galactosidase were detected in the epiblast of embryos having reached stage XIII, in the injected embryos. These positive cells were identified only in the epiblast of the zona pellucida. Later during development, at the gastrulation stage, stage 5 according to the Hamburger and Hamilton (H&H) scale, positive cells were found only in the primitive streak and the extra-embryonic germinal crescent. In the primitive streak, the cells were identified in a few aggregates mostly located in Hensen's node. At stage 13 (H&H scale), positive cells were found only in the rhomboid sinus which corresponds to the neural plate which is still open in the caudal part of the embryo. Later in embryonic development, positive cells were only found in the form of very rare isolated cells in some tissues of nervous origin, and also in the gonad rudiments.

In order to verify whether, despite the negative nature of the β-galactosidase reaction, descendants of the 9N2.5 cells had indeed colonized the tissues of late embyros in number, the presence of the ROSA-β-geo transgene was sought by PCR in DNA extracted from a whole 2-day or 4-day embryo. As is shown in FIG. 5, a band characteristic of the ROSA-β-geo transgene could be detected, demonstrating that the cells which were descendants of the transplanted 9N2.5 cells were present at least 4 days after the transplant.

Some embryos injected with 9N2.5 cells finished developing and gave rise to chicks. A search for the sequences of the ROSA-β-geo transgene was undertaken on the DNA isolated from various tissues, using the PCR technique. Thus, in two chicks which were analyzed, the presence of the transgene was revealed in the skin, the gizzard and the liver. Not all these tissues exhibited β-galactosidase activity, which demonstrates that the transgenes were present in differentiated cells derived from the transplanted 9N2.5 cells.

Conclusion

The 9N2.5 cells are therefore capable of colonizing a host embryo and of developing therein. However, expression of the ROSA-β-geo transgene remains limited to the cells very early after transplantation into embyro, and also to rare cells present in a few tissues such as the gonads or the nervous system. Given the observations made on the 9N2.5 cells in culture, it is reasonable to imagine that the expression of the ROSA-β-geo transgene in the cells in vivo is limited to the cells which have not yet committed to differentiation.

All of these data obtained in vitro and in vivo from the 9N2.5 cells lead to the supposition that the ROSA-β-geo transgene is inserted into a locus of the genome of the cells, the transcriptional activity of which is specific for ES cells in the pluripotent state.

### Example 4

### Proliferation of the 9N2.5 Cells In Vivo

In order to analyze whether the 9N2.5 cells were capable of proliferating in certain compartments of the embryo, two injected embryos were sampled after incubation for 7 days. The embryos were arbitrarily cut up into 3 sections: the head, the trunk including the upper limb rudiments, and the tail including the lower limb rudiments. These sections were dissociated in pronase and the cell suspension was seeded in culture according to the method of culturing described by Pain et al. (1996). Selection with G418 at 250 μg/ml was carried out for 6 days. A few loci of resistant cells appeared in all the cultures, but the frequency of these loci was much higher in the cultures seeded from the posterior section of the embryos. The G418 resistant cells derived from this culture were subcultured in order to be amplified, 7 days after initial seeding. Some of these parallel cultures were tested, positively, for the expression of β-galactosidase activity. This approach made it possible, for one of the 2 embryos tested, to maintain, amplify and even freeze, in viable form, cells which are positive for β-galactosidase and resistant to G418 and which exhibited a morphology identical to that of the injected 9N2.5 cells. The cells derived from the second embryo, although positive for β-galactosidase activity, proliferated only slowly and could not be sufficiently amplified.

Conclusion

These results therefore show that some 9N2.5 cells are capable of maintaining themselves in the form of ES cells in certain regions of the embryo. These cells probably correspond to the rare β-galactosidase-positive cells identified on the sections of embryos injected with the 9N2.5 cells (see above). With regard to their location in the posterior section of the embryo, it may be suggested that some of the cells which conserve the characteristics of the 9N2.5 cells in vivo correspond to EG cells as described in mice and in humans (Matsui et al. 1992, Shamblott et al. 1998). EG cells are germinal cell precursor cells which have pluripotency properties and cytological characteristics very close to those of ES cells.

### Example 5

### Use of the 9N2.5 Cells for Screening for Substances

The 9N2.5 cells strongly express β-galactosidase when they are in an undifferentiated state. This expression is lost when differentiation is induced. This property can be taken advantage of to test various differentiation-inducing or -promoting molecules or to test non-inducing molecules. The 9N2.5 cells can thus be used as a test support for identifying batches of serum suitable for culturing ES cells or for differentiation thereof. For this, the cells are seeded in a medium identical to that used for maintaining the parenteral cells. In this medium, the reference serum is replaced with the various sera to be tested, optionally at various concentrations. The seedings are carried out at very low density (2×10⁴ cells per 35 mm dish) and the cells are cultured for 4 days. The cells are then fixed, and stained to reveal β-galactosidase activity, and the number of positive loci is estimated. The number of positive loci is directly related to the ability of the serum to maintain the self-renewal of ES cells. This example can be extended to test various substances, which may be natural or synthetic.

Conclusion

The 9N2.5 cell can be used to screen for substances based on their ability to induce self-renewal or differentiation of ES cells in culture.

### Example 6

### Identification of the Locus of Integration of the ROSA-β-geo Transgene in the 9N2.5 ES Cells

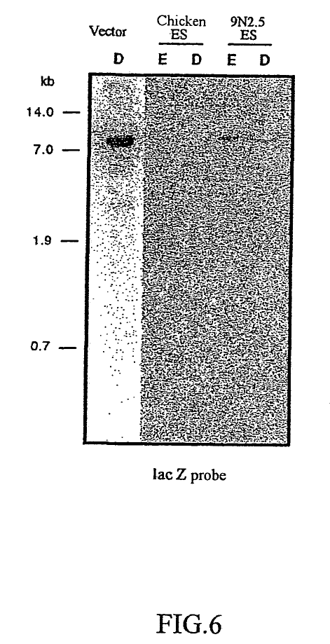

In a first approach toward identifying the locus of integration of the ROSA-β-geo transgene in the 9N2.5 cells, the genomic DNA of the 9N2.5 cells was analyzed by the Southern blotting technique. The 9N2.5 cell DNA was digested with the EcoRI restriction enzyme or with the DraI enzyme which each cleave the ROSA-β-geo transgene only at a unique site. After electrophoretic migration with digested DNA, the filters were hybridized with a probe specific for the LacZ fragment. As is shown in FIG. 6, a single band was identified under these conditions in each of the digestions performed. No band was identified in the DNA of normal chicken ES cells not containing the ROSA-β-geo transgene. These results demonstrated that, in 9N2.5 cells, a single copy of the ROSA-β-geo transgene is integrated.

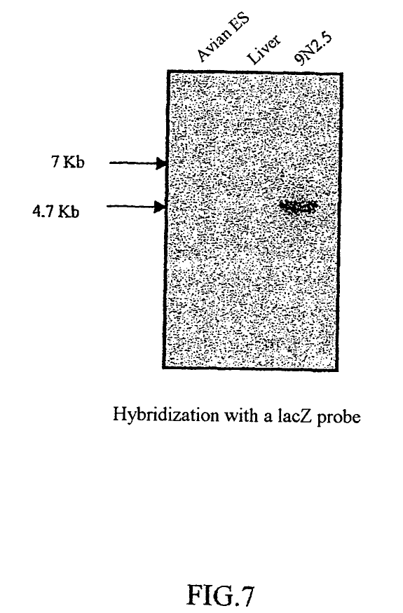

Second, the size of the mRNA transcribed from the transgene was analyzed. RNA from 9N2.5 cells was analyzed by Northern blotting with a LacZ probe. As is shown in FIG. 7, a single transcript 4.7 kb in size was revealed. This transcript is not present in the RNA of normal ES cells. Given the expected length of the sequence which should be transcribed from the ROSA-β-geo transgene, namely 3.9 kb, it must be presumed that the transcript revealed in the 9N2.5 cells contains approximately 0.8 kb of sequences derived from the cellular gene into which the transgene is inserted. These cellular sequences may be located on the mRNA either in the 5′ position or in the 3′ position, or be distributed on both sides of the sequence transcribed from the ROSA-β-geo transgene. To search for them in the 5′ region, the 5′-RACE technique using the Marathon kit from the company Clontech was employed.

A complementary DNA strand was synthesized, from 9N2.5 cell RNA, using a primer specific for LacZ region, a primer of sequence (SEQ ID No. 5).

After synthesis of the second strand complementary to this first strand, the double-stranded complementary DNA was ligated to the linker provided in the Marathon kit, the sequence of which is SEQ ID No. 6. The entire fused sequence was then amplified by the PCR technique using the primers SEQ ID No. 7 and SEQ ID No. 8.

The amplification was carried out on a Perkin Elmer 2400 machine under the following conditions: 94° C. for 30 seconds, then 5 cycles at 94° C. for 5 seconds each, then 4 minutes at 72° C., then 5 cycles at 94° C. for 5 seconds each, then 4 minutes at 70° C., then 25 cycles at 94° C. for 5 seconds each, then 4 minutes at 68° C. A 400 base pair amplification product was identified. This fragment, called F1, was cloned into a plasmid so as to be amplified, and then its exact sequence was determined. We then investigated sequences located downstream of the F1 sequence on the mRNA transcribed in the ES cells using the RT-PCR technique. For this, normal ES cell RNA was used as matrix to synthesize a complementary DNA by priming using a primer P3 of sequence SEQ ID No. 9.

The single-stranded complementary DNA was then amplified by PCR using the primers SEQ ID No. 10, which corresponds to the 5′ sequence of the fragment initially amplified by the 5′-RACE technique, and SEQ ID No. 11.

A fragment, called C1, was thus amplified and then cloned into a plasmid. The exact sequence of C1 was determined (SEQ ID No. 12).

In order to confirm that the C1 sequence is indeed in the mRNAs which also carry the LacZ sequence in the 9N2.5 cells, an amplification by RT-PCR was carried out on the mRNAs derived from these cells using the respective primers P4 (SEQ ID No. 13), which is specific for the fragment C1, and LacZB (SEQ ID No. 8), which is specific for the LacZ sequence.

A 331 base pair fragment was identified. The size of this fragment corresponds to that expected, which indicates that the C1 sequence and the LacZ sequence are indeed on the same mRNA. Confirmation was thus provided that the C1 sequence must be specific for the cellular gene into which the ROSA-β-geo transgene is inserted. This gene was called ens-1 (embryonic normal stem cell gene).

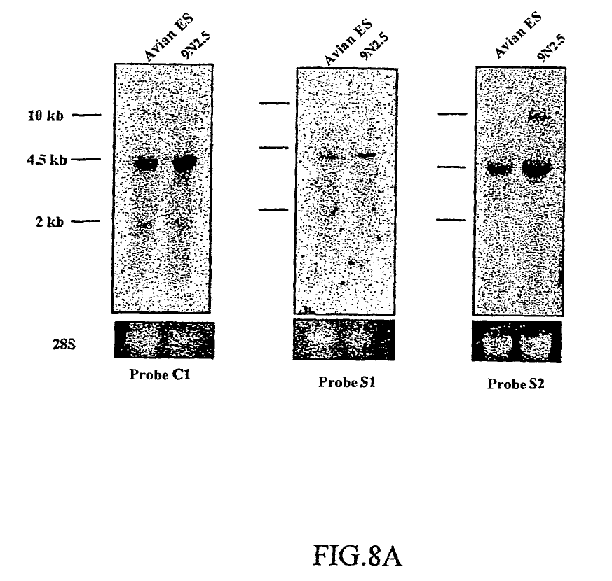

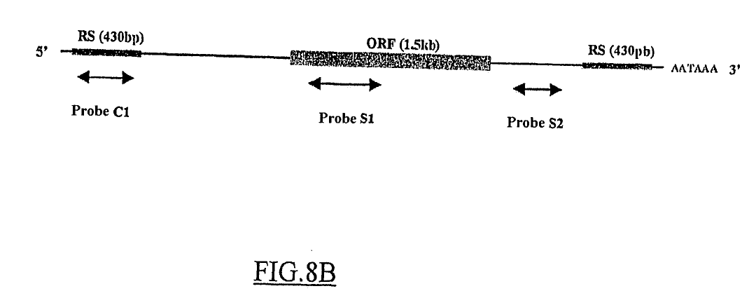

In order to verify that the ens-1 gene indeed produces a messenger RNA, the RNAs of normal chicken ES cells were analyzed by the Northern blotting technique using the C1 probe. As is shown in FIG. 8.A, the C1 probe identifies a major RNA close to 4.7 kb in size and also two RNAs very weakly labeled of approximately 10 kb and 2 kb, respectively.

Based on the C1 sequence, the cloning of the complete mRNA transcribed from the ens-1 gene was undertaken.

For this, a cDNA library constructed from polyadenylated RNA isolated from chicken ES cells was screened with probes prepared from the fragment C1.

A 4.2 kpb complementary DNA was isolated. In order to verify whether this cDNA is indeed representative of the mRNA transcribed from the ens-1 gene, two nucleotide probes were prepared, S1 and S2 respectively, corresponding to two different fragments of the CDNA, located downstream of the C1 sequence. These two probes were used to identify, by the Northern blotting technique, the corresponding RNAs isolated from normal chicken ES cells. As is shown in FIG. 8.A, these two probes identify an RNA close to 4.5 kb in size, identical to that of the major RNA identified previously with the C1 probe. As is shown later, the pattern of expression of this RNA identified with the two probes S1 and S2 is identical to that of the major RNA identified with the C1 probe in normal ES cells.

All of these data very strongly suggest that the C1, S1 and S2 probes recognize the same ens-1 mRNA in normal chicken ES cells.

The sequence of ens-1 mRNA is given in SEQ ID No. 1, and the structure of the cDNA is given in FIG. 8.B. Analysis of this sequence reveals a very long reading frame possibly encoding a protein of 490 amino acids, the sequence of which is given by SEQ ID No. 2. It should be noted that the C1 sequence is polymorphic and that that obtained from the cDNA clone, and given in SEQ ID No. 1, is slightly different from that obtained previously by 5′-RACE (SEQ ID No. 12).

In order to verify whether the ens-1 gene indeed corresponds to the gene into which the ROSA-β-geo transgene is inserted in the 9N2.5 cells. The pattern of expression of the ens-1 gene during chicken embryonic development and during differentiation of chicken ES cells in culture was analyzed using the Northern blotting technique.

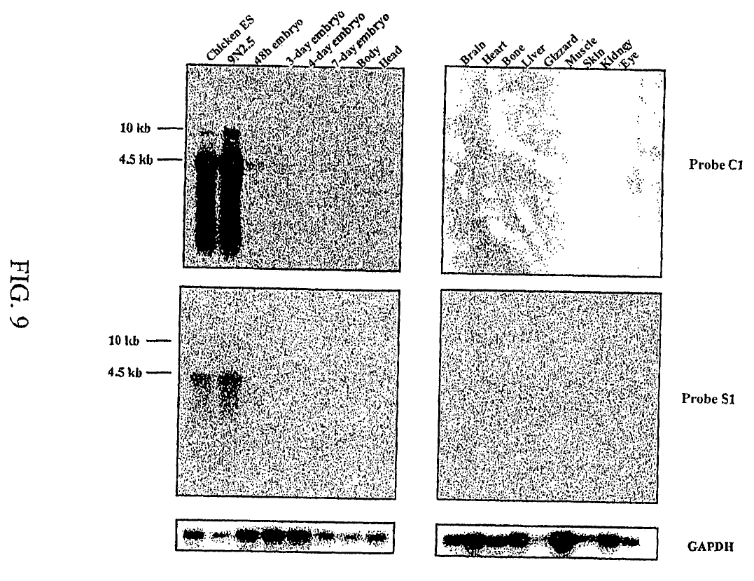

As is shown in FIG. 9, the C1 probe and the S1 probe identify the same 4.5 kb RNA in the RNAs extracted from normal 48-hour chicken embryo. The strength of the signal greatly decreases in the RNAs extracted from older embryos, such as 3-day and 4-day embryos. The signal disappears in the RNAs extracted from 7-day or 8-day embryos. It is zero in the RNAs extracted from various chick tissues such as the liver, muscle, gizzard, brain, heart, eye, bone or skin.

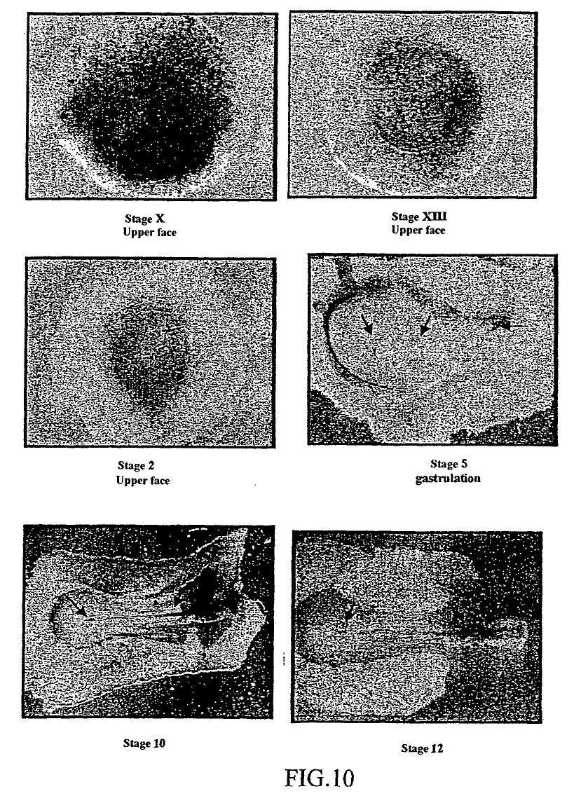

In order to determine more precisely the pattern of expression of the ens-1 gene during the first stages of development of the chicken embryo, the ens-1 mRNAs were sought using the in situ hybridization technique on whole embryo. The results are given in FIG. 10. A very strong signal was observed in the zona pellucida of stage X and XIII embryos (E-G&K scale). In stage 2 (H&H scale) embryos, the signal was found only in the zona pellucida with strong dominance in the region of the primitive streak. At stage 5 (H&H scale), the signal was found in Hensen's node and in the rostrocaudal region of the primitive streak, and also in very pronounced form in the germinal crescent positioned in the anterior portion of the embryo. At more advanced stages of embryonic development, no significant signal is detected. The same patterns of expression were observed with the C1 and S1 probes.

Conclusion

The ens-1 gene exhibits an expression specific for undifferentiated chicken ES cells and very early stages of embryogenesis. Expression of the gene becomes very weak, or even undetectable, after gastrulation has finished.

The ens-1 gene therefore constitutes a very specific marker for undifferentiated embyronic cells, whether the cells are present in the embryo, or are maintained in this state in culture in vitro. The ens-1 gene is also specific for the cells of the germinal crescent and therefore for the gamete precursor cells.

### Example 7

### Conservation of the ens-1 Gene in the Course of Evolution

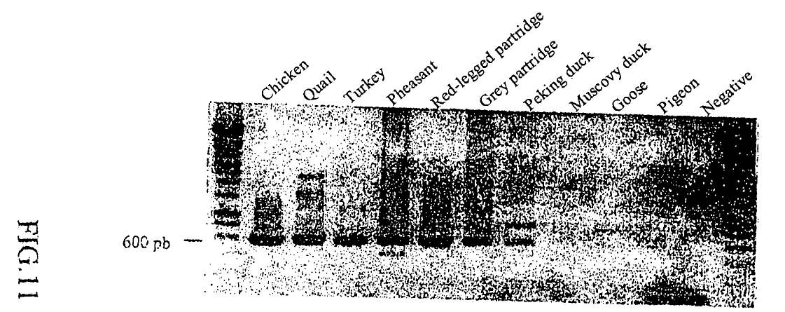

In order to analyze the degree of conservation of the ens-1 gene in the course of evolution, a probe specific for the chicken ens-1 gene was used to hybridize the genomic DNA from various animal species, using the Southern blotting technique (not shown). The technique of nucleic acid sequence amplification by PCR between two primers specific for the ens-1 gene (SEQ ID No. 14 and SEQ ID No. 15), using the protocol: 96° C. 3 minutes, (96° C. 30 s, 62° C. 30 s, 72° C. 30 s, 10 cycles), (96° C. 30 s, 57° C. 30 s, 72° C. 30 s, 10 cycles), (96° C. 30 s, 52° C. 30 s, 72° C. 30 s, 20 cycles), was also used. The results given in FIG. 11 show that homologous sequences are found only in the order Galliformes (chicken, quail, turkey, pheasant, red-legged partridge, grey partridge). It should be noted that no homolog for ens-1 is found in mammals (not shown).

### Example 8

### Identification in the ens-1 Gene of a Transcription Promoter Sequence, the Activity of which is Specific for Embryonic Stem Cells

The ens-1 gene was thus identified as being a gene specifically expressed in chicken embryonic stem cells. A promoter region, the transcriptional activity of which is specific for undifferentiated chicken ES cells, was identified in the ens-1 gene. The applications are considerable since this thus provides a genetic tool which would make it possible to target the expression of a transgene specifically in embryonic stem cells and probably also in chicken embyros at the stage preceding gastrulation.

The presence of repeat sequences at the end of the ens-1 transcript suggested that these sequences are related to retroviral LTR (long terminal repeat) sequences. Retroviral LTRs are regionalized into three sections, U3, R and U5 (in the 5′-3′ direction), respectively. In the retroviral genome, the U3 region is capable of activating transcription, sometimes with tissue-specific control. In retroviral messenger RNAs, a copy of the R-U5 sequences is found in the 5′ position and a copy of the U3-R sequences is found in the 3′ position.

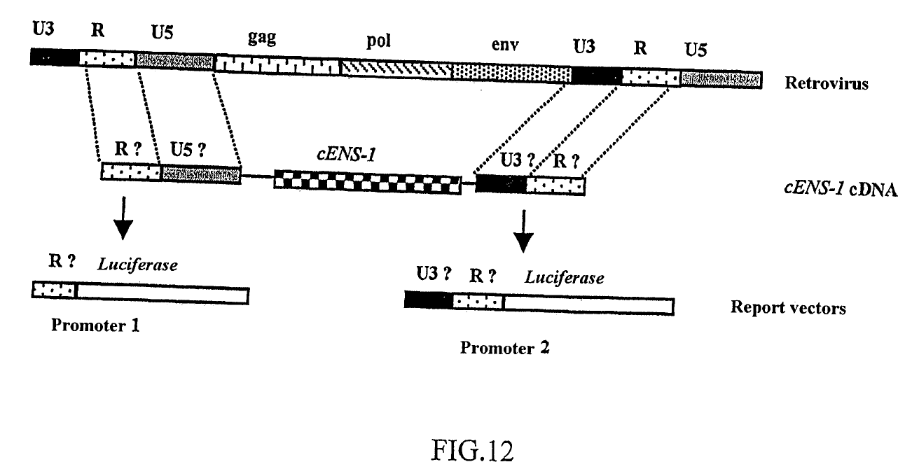

By analogy with the structure of retroviral LTRs, the regions possibly corresponding to the retroviral U3, R and U5 regions were identified in the messenger RNA of the ens-1 gene. The sequence identified as being repeated at the two ends of the ens-1 transcript corresponds to the R region and the sequence which would correspond to the U3 region is located between the 3′ end of the coding sequence for ens-1 and the 5′ end of R (FIG. 12).

To test the promoter activity of the R and U3-R regions of the ens-1 gene, these regions were cloned, in the two possible orientations, sense and antisense, upstream of the firefly luciferase reporter gene, so as to obtain, respectively, the vectors called promoter 1 and promoter 2, respectively sense (S) and antisense (AS) (FIG. 12). These constructs were transfected into various cell lines, including chicken 9N2.5 stem cells, with the vector PRL-CMV (Promega) containing the luciferase gene of the sea pansy Renilla, under the control of the cytomegalovirus promoter, as an internal control for transfection efficiency.

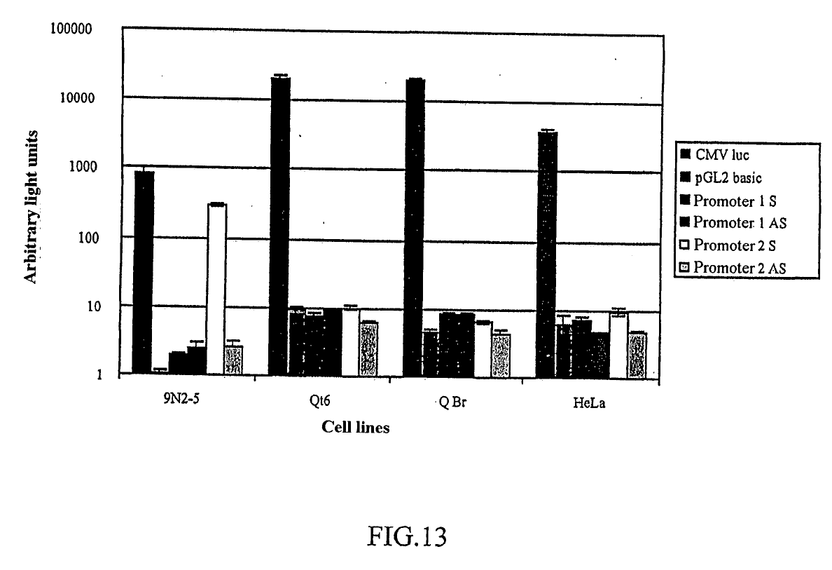

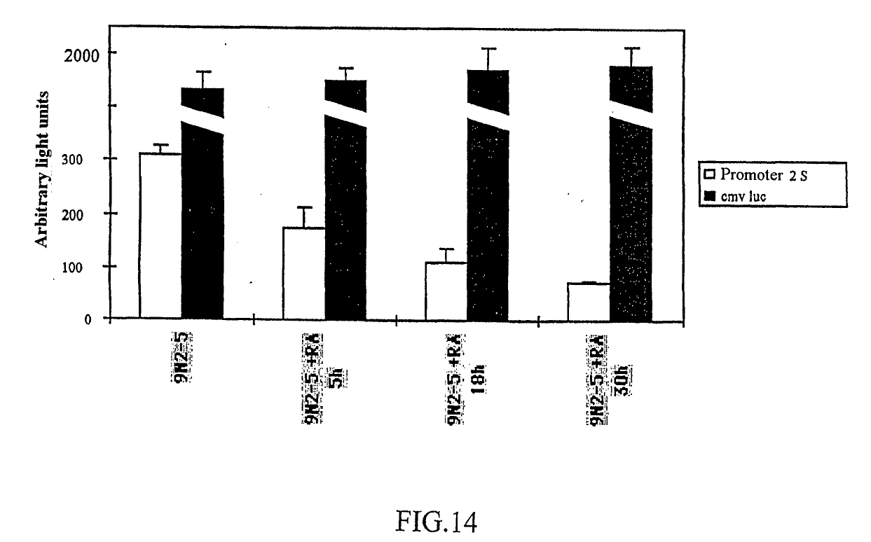

Transfection of the various vectors promoter 1 and promoter 2 into the 9N2.5 cells and measurement of luciferase activity standardized using the internal control made it possible to identify transcriptional activity for the U3 region of the promoter 2S vector in chicken embryonic stem cells (9N2.5 cells) whereas the R region shows no significant activity (FIG. 13). The activity of the promoter is, on the other hand very low in the various other cell lines tested (Qt6 quail fibroblasts, QBr quail epithelial cells or human epithelial cells). In addition, the promoter 2S vector was transfected into 9N2.5 embryonic stem cells induced to differentiate by treatment with retinoic acid. Measurement of luciferase activity in the cells at various times after treatment with retinoic acid shows that the transcriptional activity of the promoter decreases in the course of embryonic stem cell differentiation, whereas the activity of the control promoter (CMV) remains high (FIG. 14).

All of these results show that there exists, 3′ of the coding sequence of the ens1 gene, a region possessing transcription promoter activity, and that this transcriptional activity is specific for undifferentiated chicken embryonic stem cells.

Using the 5′ RACE technique on the promoter 2S vector, it was possible to determine a transcription initiation site on the sequence of the ens-1 cDNA (SEQ ID No. 1), and also a sequence of the TATA promoter type upstream of this transcription initiation site. The promoter corresponds to nucleotides 3111-3670 of SEQ ID No. 1.

Deposition of Biological Material

The 9N2.5 cell line was deposited, on May 11, 2000, with the Collection Nationale de Cultures des Microorganismes (CNCM) [National Collection of Cultures and Microorganisms], 25 rue du Docteur Roux, 75724 Paris Cedex 15, France, according to the provisions of the Treaty of Budapest, under the identification number I-2477, and corresponds to the line of chicken embyronic stem cells into which the ROSA-β-geo transgene linearized with DraI was introduced by electroporation, and which were isolated after selection with G418, and based on their β-galactosidase activity, as described in Example 1.

The cells which can be used to culture the 9N2.5 cells (STO mouse fibroblasts) were also deposited with the CNCM, on May 11, 2000, under the number SH-2477.

### REFERENCES

Buckholz, (1993), Curr. Op. Biotechnology 4, 538.

Carter, (1993), Curr. Op. Biotechnology 3, 533.

Duck et al. (1990), Biotechniques, 9, 142.

Edwards and Aruffo (1993), Curr. Op. Biotechnology 4, 558.

Epstein (1992), Médecine/Sciences, 8, 902.

Etches et al. (1996), Science 76, 1075-1083.

Eyal-Giladi and Kovak (1976), Dev. Biol. 151, 575-585.

Freidrich and Soriano (1991). Genes & Development 5, 1513-1523.

Guatelli et al. (1990), Proc. Natl. Acad. Sci. USA 87: 1874.

Kemler et al. (1981). J. Embryol. Exp. Morph. 64, 45-60.

Kievitis et al. (1991), J. Virol. Methods, 35, 273.

Köhler and Milstein. (1975) Nature 256, 495.

Kwoh et al. (1989), Proc. Natl. Acad. Sci. USA, 86, 1173

Landegren et al. (1988) Science 241, 1077.

Luckow (1993), Curr. Op. Biotechnology 4, 564.

Matsui et al. (1992). Cell 70, 841-847.

Matthews et al. (1988), Anal. Biochem., 169, 1-25.

Miele et al. (1983), J. Mol. Biol., 171, 281.

Neddleman and Wunsch (1970) J. Mol. Biol. 48: 443

Olins and Lee (1993), Curr. Op. Biotechnology 4: 520.

Pain et al. (1996). Development 122, 2339-2348.

Pain et al. (1999). Cells Tissues Organs 165, 212-219.

Perricaudet et al. (1992). La Recherche 23: 471.

Pearson and Lipman (1988) Proc. Natl. Acad. Sci. USA 85: 2444

Prowse and Greider (1995). Proc. Natl. Acad. Sci. USA 92, 4818-4822.

Rohlmann et al. (1996) Nature Biotech. 14: 1562.

Rolfs, A. et al. (1991), Berlin: Springer-Verlag.

Rosner et al. (1990). Nature 354, 686-692.

Sambrook et al. (1989) Molecular cloning: a laboratory manual. 2^{nd} Ed. Cold Spring Harbor Lab., Cold Spring Harbor, N.Y.

Segev, (1992), Kessler C. Springer Verlag, Berlin, New-York, 197-205.

Shamblott et al. (1998). Proc. Natl. Acad. Sci. USA 95, 13726-13731.

Smith and Waterman (1981) Ad. App. Math. 2: 482

Solter and Knowles (1978) Proc. Natl. Acad. Sci. USA 75, 5565-5569.

Stewart and Yound (1984), Solid phase peptides synthesis, Pierce Chem. Company, Rockford, 111, 2^{nd} ed., (1984).

Strickland et al. (1980). Cell 21, 347-355.

Temin, (1986) Retrovirus vectors for gene transfer. In Kucherlapati R., ed. Gene Transfer, New York, Plenum Press, 149-187.

Walker (1992), Nucleic Acids Res. 20: 1691.

1. A purified or isolated nucleic acid characterized in that it comprises a nucleic acid sequence chosen from the group of following sequences:
a) SEQ ID No. 1, or the fragment corresponding to nucleotides 1409-2878 of SEQ ID No. 1;
b) the sequence of a fragment of at least 15 consecutive nucleotides of a sequence chosen from SEQ ID No. 1, in particular the fragment corresponding to nucleotides 3111-3670 of SEQ ID No. 1;
c) a nucleic acid sequence having a percentage identity of at least 80%, after the optimal alignment, with a sequence defined in a) or b), said sequence not being defined by nucleotides 2308-2927 or 3094-3753 of SEQ ID No. 1;
d) a nucleic acid sequence which hybridizes, under high stringency conditions, with a nucleic acid sequence defined in a) or b), said sequence not being defined by nucleotides 2308-2927 or 3094-3753 of SEQ ID No. 1;
e) the complementary sequence or the RNA sequence corresponding to a sequence as defined in a), b), c) or d).

2. The purified or isolated nucleic acid as claimed in claim 1, characterized in that it comprises or consists of SEQ ID No. 1, the complementary sequence or the RNA sequence corresponding to one of these sequences.

3. A purified or isolated nucleic acid, characterized in that it encodes a polypeptide which has a continuous fragment of at least 200 amino acids of the protein SEQ ID No. 2.

4. An isolated polypeptide, characterized in that it comprises a polypeptide chosen from:
a) a polypeptide corresponding to SEQ ID No. 2;
b) a variant polypeptide of a polypeptide of sequence defined in a);
c) a polypeptide homologous to a polypeptide defined in a) or b), comprising at least 80% homology with said polypeptide of a);
d) a fragment of at least 15 consecutive amino acids of a polypeptide defined in a), b) or c);
e) a biologically active fragment of a polypeptide defined in a), b) or c).

5. The polypeptide as claimed in claim 4, characterized in that it consists of a sequence chosen from SEQ ID No. 2, or a sequence having at least 80% homology with this sequence after optimal alignment.

6. A cloning and/or expression vector comprising a nucleic acid as claimed in one of claims 1 to 3 or encoding a polypeptide as claimed in either of claims 4 and 5.

7. A host cell, characterized in that it is transformed with a vector as claimed in claim 6.

8. A host cell containing a nucleic acid as claimed in one of claims 1 to 3, characterized in that it is an avian ES cell also modified by introducing an exogenous gene, said exogenous gene being expressed only and specifically when said cell is maintained in the pluripotent state.

9. The cell as claimed in claim 8, characterized in that said exogenous gene is a reporter gene.

10. The cell as claimed in claim 9, characterized in that said reporter gene is chosen from lacZ, GFP, luciferase, ROSA-β-geo and a gene for resistance to an antibiotic.

11. A host cell containing a nucleic acid as claimed in one of claims 1 to 3, characterized in that it is an avian cell also modified by introducing an exogenous nucleic acid, said exogenous nucleic acid being integrated into said nucleic acid as claimed in one of claims 1 to 3.

12. The cell as claimed in claim 11, characterized in that said exogenous nucleic acid is a gene of therapeutic interest, optionally preceded by a spatio-temporal promoter and/or by terminator sequences.

13. The cell as claimed in claim 11, characterized in that said exogenous nucleic acid is a genetic marker.

14. The cell as claimed in one of claims 8 to 13, characterized in that said bird belongs to the order Galliformes.

15. The cell as claimed in claim 14, characterized in that said bird is a chicken or a quail.

16. The cell as claimed in either of claims 14 and 15, characterized in that said reporter gene is integrated under the control of the promoter of the ens-1 gene.

17. The cell as claimed in either of claims 14 and 15, characterized in that it is a 9N2.5 cell, deposited with the Collection Nationale [lacuna] des Microorganismes on May 11, 2000, under the identification number I-2477.

18. A differentiated avian cell, characterized in that it derives from an ES cell as claimed in one of claims 8 to 17.

19. An animal, except for human, characterized in that it comprises a cell as claimed in one of claims 7 to 18.

20. The use of a nucleic acid sequence as claimed in one of claims 1 to 3, as a probe or primer, for detecting and/or amplifying nucleic acid sequences.

21. The use of a nucleic acid as claimed in one of claims 1 to 3, as a sense or antisense oligonucleotide.

22. The use of a nucleic acid sequence as claimed in one of claims 1 to 3, for producing a recombinant polypeptide.

23. A method for obtaining a recombinant polypeptide, characterized in that a cell as claimed in claim 7 is cultured under conditions which allow the expression of said polypeptide, and in that said recombinant polypeptide is recovered.

24. A recombinant polypeptide, characterized in that it is obtained using a method as claimed in claim 23.

25. A monoclonal or polyclonal antibody, characterized in that it selectively binds a polypeptide as claimed in one of claims 4, 5 and 24.

26. A method for detecting a polypeptide as claimed in one of claims 4, 5 and 24, characterized in that it comprises the following steps:
a) bringing a biological sample into contact with an antibody as claimed in claim 25;
b) demonstrating the antigen-antibody complex formed.

27. A kit of reagents for carrying out a method as claimed in claim 26, characterized in that it comprises:
a) a monoclonal or polyclonal antibody as claimed in claim 25;
b) optionally, reagents for constituting a medium suitable for the immunoreaction;
c) the reagents for detecting the antigen-antibody complex produced during the immunoreaction.

28. A method for determining the pluripotent nature of an avian ES cell, characterized in that the presence of a product of expression of the gene corresponding to SEQ ID No. 1, or of the mRNA of SEQ ID No. 1, is determined.

29. The method as claimed in claim 28, characterized in that the mRNA of SEQ ID No. 1 is detected by Northern blotting or by RT-PCR using a probe or primers, by the use as claimed in claim 20.

30. The method as claimed in claim 28, characterized in that the presence of the protein SEQ ID No. 2 is detected, for example using an antibody as claimed in claim 25.

31. A method for classifying a bird as belonging to the order Galliformes, characterized in that the presence of a nucleic acid as claimed in one of claims 1 to 3 is detected in the genome of said bird.

32. A method for determining the presence of a sample originating from a bird of the order Galliformes in a food sample, characterized in that the presence of a nucleic acid as claimed in one of claims 1 to 3 is detected in said sample.

33. A DNA chip, characterized in that it contains a nucleic acid sequence as claimed in one of claims 1 to 3.

34. A protein chip, characterized in that it contains a polypeptide as claimed in one of claims 4, 5 and 24, or an antibody as claimed in claim 25.

35. A method for detecting and/or assaying a nucleic acid according to one of claims 1 to 3, in a biological or food sample, characterized in that it comprises the following steps:
a) bringing said sample into contact with a polynucleotide as claimed in one of claims 1 to 3, which is labeled;
b) detecting and/or assaying the hybrid formed between said polynucleotide and the nucleic acid of said sample.

36. A method for detecting and/or assaying a nucleic acid as claimed in one of claims 1 to 3, in a biological or food sample, characterized in that it comprises a step of amplification of the nucleic acids of said sample using primers chosen from the nucleic acids as claimed in either of claims 1 and 2.

37. A method for screening for a substance or for a medium capable of inducing differentiation of pluripotent cells, characterized in that it comprises the following steps:
a) maintaining ES cells as claimed in one of claims 7 to 17 in a culture medium making it possible to maintain the pluripotent phenotype;
b) adding said substance to said culture medium or replacing said culture medium with the medium to be tested;
c) determining the induction of differentiation by the absence of expression of the protein SEQ ID No. 2 or of the exogenous gene.

38. The method as claimed in claim 37, characterized in that it is carried out with cells as claimed in one of claims 8 to 17.

39. The method as claimed in claim 37 or 38, characterized in that 9N2.5 cells are used, and in that the absence of expression of β-galactosidase is detected.

40. A method for screening for a substance capable of restoring the pluripotent nature of differentiated cells, characterized in that it comprises the following steps:
a) maintaining differentiated cells in a suitable culture medium;
b) replacing said culture medium with a medium which makes it possible to maintain a pluripotent phenotype and which contains said substance to be tested;
c) determining the restoration of the pluripotent nature of said cells by the expression of the protein SEQ ID No. 2 or of the exogenous gene, in said cells.

41. The method as claimed in claim 40, characterized in that it is carried out with differentiated cells derived from cells as claimed in one of claims 8 to 17.

42. The method as claimed in claim 40 or 41, characterized in that differentiated 9N2.5 cells are used, and in that the expression of β-galactosidase is detected.

43. A medium or a substance, characterized in that it is obtained using a method as claimed in one of claims 37 to 42.

44. A compound, characterized in that it is chosen from
a) a nucleic acid as claimed in one of claims 1 to 3;
b) a polypeptide as claimed in one of claims 4, 5 and 24;
c) a vector as claimed in claim 6;
d) a cell as claimed in one of claims 7 to 17;
e) an antibody as claimed in claim 25;
f) a substance as claimed in claim 43,
as a medicinal product.

45. The use of a nucleic acid corresponding to nucleotides 3111-3670 of SEQ ID No. 1, as a promoter of a gene of interest, for specific expression of said gene of interest in avian pluripotent cells.
